(19) United States
(12) Patent Application Publication
(10) Pub. No.: US 2006/0115863 A1
(43) Pub. Date: Jun. 1, 2006

(54) Inositol phosphate detection assays

(75) Inventors: Robert Bostwick, Wilmington, DE (US); Deborah Hartman, Wilmington, DE (US); Jay Lie Liu, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/539,544

(22) PCT Filed: Dec. 17, 2003
(86) PCT No.: PCT/SE03/01978

§ 371 Date: Jun. 16, 2005

Related U.S. Application Data
(60) Provisional application No. 60/434,264, filed on Dec. 18, 2002.

(51) Int. Cl. G01N 33/537; C12Q 1/42; G01N 33/00

(52) U.S. Cl. 435/7.92; 435/21; 436/104

(57) ABSTRACT

Assays for detecting or measuring inositol phosphate, for detecting or measuring signaling pathways, for detecting or measuring the activity of phospholipase C-linked receptors and/or their cognate pathways, for detecting or measuring the activity of inositol kinases and inositol phosphatases, and for screening for compounds that modulate the activity of signaling pathways, receptors and enzymes.

### FIELD OF THE INVENTION

The present invention relates to methods of detecting or measuring inositol phosphate, assays for detecting or measuring the activity of signaling pathways, phospholipase C-linked receptors, kinases, and phosphatases, and assays for screening for compounds that modulate signaling pathways and the activity of receptors and enzymes.

### BACKGROUND

The regulation of phosphoinositide (PI) metabolism is a key component in cell signaling networks and pathways (Micheu, 1992, Trends Biochem. Sci., 17:274-276; Payrastre et al., 2001, Cell Signal, 13:377-387). A great number of hormones, growth factors, cytokines and neurotransmitters communicate with cell interior via receptors coupled to phospholipase C (PLC). Activation of PLC-β through the heterotrimeric G protein pathway or PLC-γ through the tyrosine kinase pathway leads to an increase in the hydrolysis of phosphatidylinositol 4,5-bisphosphate (PIP2) and the generation of two ubiquitous second messengers, inositol 1,4,5-trisphosphate (IP3) and diacylglycerol, which then trigger the subsequent signaling cascades and cellular events (Martin, 1991, Pharmacol. Ther., 49:329-345). While this system has been under intensive studies for decades, no significant improvement has been made in the methodology of phosphoinositide hydrolysis measurement. Currently, the most widely used method was developed 20 years ago by Berridge which employs solvent extraction and anion-exchange chromatography to separate labeled inositol phosphate from inositol and phosphoinositide lipid after receptor stimulation in the presence of LiCl (Berridge et al., 1982, Biochem. J., 206(3):587-595). Although highly sensitive, this method suffers from significant limitations, such as the time-consuming and labor-intensive processing of samples, which results in low throughput and the generation of a large volume of radioactive waste.

A number of PLC-linked receptors are molecular targets for therapeutic interventions. Since the increase in phosphoinositide hydrolysis is directly linked to receptor activation, its measurement has been frequently used as a functional assay to study the interactions of pharmacological agents with their receptors. In addition, the measurement of phosphoinositide hydrolysis can be used to identify novel agonists, antagonists, or modulators acting at receptors that are coupled to PLC activation. Recent advances in generating a large number of compounds through modern medicinal chemistry technologies together with ever increasing number of novel molecular targets identified from genomic efforts have increased the pressure to develop methodologies to enable rapid evaluation of compound libraries to identify lead chemical structures.

Inositol monophosphatase (IMPase), the enzyme that dephosphorylates inositol monophosphates to regenerate inositol, is associated with mental disease. Specifically, IMPase activity in cerebrospinal fluid (CSF) is significantly increased in patients suffering from depression, bipolar disease, and schizophrenia, and lithium treatment can return IMPase activity to normal levels in bipolar patients (Atack, 1996, Brain Res. Rev., 22:183-190; Atack et al., 1998, Biol. Psychiatry, 44:433-437). Compounds that can inhibit IMPase are useful in the treatment of bipolar disorders. In addition, CSF IMPase activity serves as a marker for mental illness.

Inositol-1-phosphate synthase (INPS) catalyses the addition of phosphate to inositol, and is responsible for the production of inositol phosphate in archaea and eubacteria (Bachhawat et al., 2002, Trends Genet., 16:111-113). INPS is a key enzyme involved in the phosphatidylinositol (PI) biosynthetic pathway (Norman et al., 2002, Structure, 10:393402). The enzyme is known to be overexpressed in isoniazid resistant strains of Myobacterium tuberculosis, and has been shown to be important for its virulence. Since PI is essential for M. tuberculosis viability, INPS is a target for antimycobacterial agents and drugs.

Ideally, receptor-stimulated PI hydrolysis would be monitored as the rate of production of IP₃ in the absence of degradation, i.e., in a manner analogous to the measurement of cAMP accumulation in the presence of phosphodiesterase inhibitors. However, the metabolism of IP₃ occurs rapidly in most cells and specific cell-permeant inhibitors of enzymes that metabolize IP₃ have yet to be identified (Fisher, 1995, Eur. J. Pharmacol., 288:231-250; Wojcikliewicz et al., 1993, Trends Pharmacol. Sci, 14:279-285). As an alternative; measurement of PI hydrolysis has been performed in the presence of LiCl, which blocks inositol monophosphatase and results in the accumulation of IP₃ metabolites, most notably inositol-1-phosphate (IP₁). The assay has been commonly performed on cells pre-incubated with [³H]inositol. In these cells, phosphoinositides, including phophatidylinositol, phosphatidylinositol 4-phosphate and phosphatidylinositol 4,5-bisphosphate, become radiolabeled and hydrolysis of these lipids by PLC generates [³H]inositol phosphates, which are isolated by ion exchange chromatography and quantified by liquid scintillation counting (Berridge et al., 1982, supra; Berridge et al., 1984, Biochem. J., 222:195-201; Liu et al., 1996, J. Biol. Chem., 271:6172-6178).

The principle of Immobilized Metal Affinity Chromatography (IMAC) makes use of matrix-bound metals to purify biomaterials on the basis of their interaction with the immobilized metal ions (Porath et al., 1983, Biochemistry, 22:1621-1630; Sulkowski, 1989, Bioessays, 10:170-175; Yip et al., 1996, Methods Mol. Biol., 59:197-210; Gaberc-Porekar et al., 2001, J. Biochem. Biophys. Methods, 49:335-360). It has been used for about two decades to purify proteins, peptides and nucleic acids (Sulkowski, 1985, Trends Biotechnol., 3:1-7; Jiang et al., 1996, Anal. Biochem., 242:45-54; Haupt et al., 1996, Anal. Biochem., 234:149-154). The most common application is using Ni²⁺-column to purify polyhistidine-tagged proteins. Based on the original observation by Anderson and Porath that immobilized Fe³⁺ bound free phosphate with high affinity, Fe³⁺-IMAC has been successfully used to isolate phosphorylated peptides (Anderson et al., 1986, Anal. Biochem., 154:250-254; Li S, et al., 1999, Anal. Biochem., 270:9-14; Scanff et al., 1991, J. Chromatogr., 539:425-432). The strong interaction of the phosphate group with IDA-Fe³⁺ is believed to be due to the formation of two coordination bonds resulting in a four-member ring complex (Anderson et al., supra; Chaga et al., 1992, J. Chromatogr., 627:163-172, Muszynska et al., 1986, Biochemistry, 25:6850-6853; Holmes et al., 1997, J. Liq. Chromatogr. Rel. Technol., 20:123-142).

Historically, several approaches have been used to study PI hydrolysis, but are now rarely used, which include the measurement of the increased ³²P-labelling of either phosphatidate or PI as a secondary event of PI hydrolysis and the measurement of IP₃ by mass spectroscopy following HPLC (Fisher, supra; Wojcikiewicz et al., supra; Shears, 1992, in Inositol Phosphates and Calcium Signalling (Advances in Second Messenger and Phosphoprotein Research), Vol 26, pp 63-92, J. W. Putney, Jr. (ed.), Raven Press, New York). Recently, a ligand binding assay has been reported for the measurement of IP₃ as an index of PI hydrolysis (Viko et al., 1998, Pharmacol Toxicol, 83:23-28; Hanem et al., 1996, Mol. Cell. Biochem., 164:167-172), which employs specific receptors or binding proteins purified from adrenal gland or other tissues to capture 1,4,5-IP₃ exclusively (Guillemette et al., 1987, Proc. Natl. Acad. Sci. U.S.A, 84:8195-9199; Baukal et al., 1985, Biochem. Biophys. Res. Commun., 133:532-538; Theibert et al., 1987, Biochem. Biophys. Res. Commun., 148:1283-9). Amersham Biosciences Corp. (Piscataway, N.J.) and PerkinElmer (Boston, Mass.) have both developed a RIA kit (TRK1000 and NEK064, respectively) for this assay technique. Despite the advantages of measuring the most relevant species of inositol phosphates, the use of this method is limited by the fact that IP₃ is rapidly metabolized in the cell resulting in a transient elevation of IP₃ with a half-life less than a minute (Hughes et al., 1989, J. Biol. Chem., 264:9400-9407; Fisher et al., 1992, J. Neurochem., 58:18-38; Fisher et al., 1990, Mol. Pharmacol., 38:54-63). This requires precise control or timing of the agonist stimulation and the termination of the reaction in the scale of seconds. In addition, the IP₃ peak time is variable according to the temperature, pH, the association rate and the concentration of the agonist compound. The success of this method will depend on the future identification of cell-permeant inhibitors of the IP₃-metabolizing enzymes, 5-phosphatase and IP₃ 3-kinase.

### SUMMARY

The present invention provides methods for detecting or measuring inositol phosphate.

The present invention provides methods for detecting or measuring inositol phosphate in a sample comprising contacting the sample with an immobilized metal ion and detecting inositol phosphate as bound to the immobilized metal ion.

The present invention also provides methods for detecting or measuring inositol phosphate in a sample comprising contacting the sample with an immobilized metal ion bound to inositol phosphate, said inositol phosphate attached to a label, and detecting displacement of the inositol phosphate from the metal ion.

The present invention also provides methods for detecting activation of a signaling pathway comprising contacting a sample with an immobilized metal ion, and detecting inositol phosphate as bound to the immobilized metal ion.

The present invention also provides methods for detecting modulation of a signaling pathway comprising contacting a sample with an immobilized metal ion, and detecting inositol phosphate as bound to the immobilized metal ion.

The present invention also provides methods for identifying compounds that modulate a signaling pathway comprising, in the presence and in the absence of a test compound, contacting a sample with an immobilized metal ion; and detecting inositol phosphate as bound to the immobilized metal ion.

The present invention also provides methods for detecting activation of a phospholipase C-linked receptor and/or its pathway comprising providing cells expressing a receptor that utilizes a phospholipase C signaling pathway, contacting the cells with labeled inositol, contacting the cells with a receptor agonist, whereby labeled inositol phosphate is generated, releasing the labeled inositol phosphate from the cells, contacting the labeled inositol phosphate with an immobilized metal ion under conditions permitting inositol phosphate to bind to the metal ion; and detecting labeled inositol phosphate as bound to the immobilized metal ion, wherein bound labeled inositol phosphate is indicative of receptor and/or pathway activation.

The present invention also provides methods for identifying compounds that modulate a phospholipase C-linked receptor and/or its pathway comprising, in the presence and in the absence of a compound, providing cells expressing a receptor that utilizes a phospholipase C signaling pathway, contacting the cells with labeled inositol, contacting the cells with a receptor agonist, whereby labeled inositol phosphate is generated, releasing labeled inositol phosphate from the cells, contacting the labeled inositol phosphate with an immobilized metal ion under conditions to allow inositol phosphate to bind to the metal ion, and detecting labeled inositol phosphate as bound to the immobilized metal ion, wherein an alteration in the amount of bound labeled inositol phosphate in the presence of a compound identifies said compound as a compound that modulates the phospholipase C-linked receptor and/or its pathway.

The present invention also provides methods for detecting inositol monophosphatase activity in a sample comprising contacting the sample with labeled inositol phosphate under conditions permitting inositol monophosphatase to hydrolyze phosphate from inositol phosphate, contacting the sample with an immobilized metal ion under conditions permitting inositol phosphate to bind to the metal ion, and detecting labeled inositol phosphate as bound to the immobilized metal ion, wherein a decrease in the amount of bound labeled inositol phosphate, as compared to a control, is indicative of inositol monophosphatase activity in the sample.

The present invention also provides methods for identifying compounds that modulate inositol monophosphatase activity comprising, in the presence and in the absence of a compound, a) contacting inositol monophosphatase with labeled inositol phosphate under conditions permitting inositol monophosphatase to hydrolyze phosphate from inositol phosphate, b) contacting the reaction mixture of step a) with an immobilized metal ion under conditions permitting inositol phosphate to bind to the metal ion, and c) detecting labeled inositol phosphate as bound to the immobilized metal ion, wherein an alteration in the amount of bound labeled inositol phosphate in the presence of a compound identifies said compound as a compound that modulates inositol monophosphatase activity.

The present invention also provides methods for detecting inositol-1-phosphate synthase activity in a sample comprising contacting the sample with labeled inositol under conditions permitting inositol-1-phosphate synthase to catalyse addition of phosphate to inositol, contacting the sample with an immobilized metal ion under conditions permitting inositol phosphate to bind to the metal ion, and detecting labeled inositol phosphate as bound to the immobilized metal ion, wherein an increase in the amount of bound labeled inositol phosphate, as compared to a control, is indicative of inositol-1-phosphate synthase activity in the sample.

The present invention also provides methods for identifying compounds that modulate inositol-1-phosphate synthase activity comprising, in the presence and in the absence of a compound, a) contacting inositol-1-phosphate synthase with labeled inositol under conditions permitting inositol-1-phosphate synthase to catalyse addition of phosphate to inositol, b) contacting the reaction mixture of step a) with an immobilized metal ion under conditions permitting inositol phosphate to bind to the metal ion, and c) detecting labeled inositol phosphate as bound to the immobilized metal ion, wherein an alteration in the amount of bound labeled inositol phosphate in the presence of a compound identifies said compound as a compound that modulates inositol-1-phosphate synthase activity.

### BRIEF DESCRIPTION OF THE DRAWINGS

FIGS. 1A and 1B show binding of [³H]inositol and [³H]inositol-1-phosphate to the SPA beads loaded with different metal ions. 10 nCi of [³H]inositol or [³H]inositol-1-phosphate were incubated with 1 mg metal ion-loaded SPA beads. After vacuum filtration, the radioactivity that remained in the flow-through samples was measured by liquid scintillation counting (FIG. 1A), and the radioactivity retained on the beads was detected by SPA technology (FIG. 1B). Each data point represents the mean ±SD of triplicate samples.

FIG. 2 shows the pH-dependent binding of 10 nCi [³H]inositol-1-phosphate to 1mg Zr⁴⁺-loaded SPA beads. Each data point represents the mean±SD of triplicate samples.

FIG. 3 shows the blockade of the binding of 10 nCi [³ H]inositol-1-phosphate to 2 mg Zr⁴⁺-loaded SPA beads by pretreatment of the beads with unlabeled inositol-1-phosphate or ATP. Each data point represents the mean±SD of triplicate samples.

FIG. 4 shows the measurement of NK1-mediated PI hydrolysis in the cells stimulated with 0.1 μM Substance P or saline by using 2 mg SPA beads loaded with different metal ions. Each data point represents the mean±SD of triplicate samples.

FIG. 5 shows the titration of the amount of Zr⁴⁺-SPA beads for the measurement of 0.1 μM Substance P-stimulated PI hydrolysis in 96-well plates. Each data point represents the mean±SD of triplicate samples.

FIG. 6 shows the concentration-dependent stimulation of NK1-mediated PI hydrolysis by Substance P with or without the pretreatment of the cells with NK1 antagonist L-733060 at 10, 50 and 250 nM. The responses were measured with 2 mg/well Zr⁴⁺-SPA beads. Each data point represents the mean±SD of duplicate samples.

FIG. 7 shows the concentration-dependent stimulation of PI hydrolysis by PDGF-BB in quiescent Swiss 3 T3 cells. The responses were measured by using 2 mg/well Zr⁴⁺-SPA beads. Each data point represents the mean±SD of duplicate samples.

### DETAILED DESCRIPTION

The present invention is based upon our discovery that immobilized metal ions can be used as affinity ligands to entrap inositol phosphates. Since immobilized metal ions bind inositol phosphate, rather than inositol, inositol phosphate generated from a variety of enzymatic and biochemical reactions can be detected and/or measured and/or quantitated without any separation steps and without the use of scintillation cocktails. The methods of the present invention are broadly applicable in signal transduction research, drug discovery, and drug development, including, without limitation, analysis of clinical samples.

Our discovery can be harnessed in methods for detecting the presence of inositol phosphate in a sample. These assays can be used to detect and/or measure and/or quantitate inositol phosphate for monitoring its formation and degradation by biological or chemical synthetic pathways, for monitoring the activity of enzymes and/or signaling pathways involved in inositol phosphate metabolism, and for screening for compounds that modulate the activities of such enzymes and/or signaling pathways. For example, the assays of the present invention can be used to measure the yield of synthetic reactions and processes that generate or degrade inositol phosphate. The detection methods of the present invention are based upon using immobilized metal ions to bind inositol phosphate, such that the bound inositol phosphate can then be detected and/or measured and/or quantitated.

The present invention provides methods for detecting inositol phosphate in a sample, by contacting the sample with an immobilized metal ion, and detecting inositol phosphate as bound to the immobilized metal ion. The inositol phosphate that is bound to the immobilized metal ion can be detected in many ways known to the art. For example, the inositol phosphate can be detected by virtue of an attached label. Where the inositol phosphate in the sample is already attached to a label, the bound inositol phosphate is detected by detection of the label. Where the inositol phosphate in the sample is not already attached to a label, it can be detected by measuring the displacement of labeled inositol phosphate that has been bound to the immobilized metal ion.

In some embodiments, the present invention provides methods for detecting inositol phosphate in a sample, where the methods comprise contacting the sample with an immobilized metal ion under conditions permitting inositol phosphate to bind to the metal ion, and measuring inositol phosphate bound to the immobilized metal ion, wherein inositol phosphate bound to the immobilized metal ion is indicative of inositol phosphate in the sample.

The sample that is analyzed can be from any source, including, but not limited to, biological, medical, and in vivo or in vitro reaction mixtures. The sample can include cells or cellular components (such as membrane fractions) or cell-free systems in which biological pathways are occurring to result in the generation or degradation of inositol phosphate. The methods of the present invention can therefore be used to monitor the activity of natural and/or synthetic and/or recombinant enzymes, proteins or systems that generate or degrade inositol phosphate.

Any method of detecting and/or measuring and/or quantitating bound inositol phosphate can be used. For example, the bound inositol phosphate can be detected on the basis of an attached or otherwise incorporated detectable label, or via competition with pre-bound labeled inositol phosphate. Such competition assays can be used to monitor inositol phosphate in samples where the inositol phosphate has no label. Decreasing amounts of bound, labeled inositol phosphate would be indicative of the presence of inositol phosphate in the sample.

Our discovery can be harnessed in assays designed to monitor receptor-mediated phosphoinositide hydrolysis and in assays designed to monitor and/or detect and/or measure and/or quantitate the activity of receptors or enzymes or signaling pathways that directly or indirectly phosphorylate inositol or enzymes or signaling pathways that directly or indirectly hydrolyze inositol phosphate. As used herein, the term “signaling pathway” includes pathways regulated or activated by enzymes or receptors, including but not limited to, phospholipase C-linked receptors. The present invention provides assays for measuring the activation and activity of a variety of phospholipase C-linked receptors. We have used IMAC-SPA beads to measure the inositol phosphate responses mediated by the G protein-coupled neurokinin NK1 receptor and the platelet-derived growth factor (PDGF) cytokine receptor. The present invention also provides assays for detecting the presence of and measuring the activity of inositol monophosphatase and inositol-1-phosphate synthase. The present invention also provides assays for identifying compounds that modulate the activities of receptors or enzymes or signaling pathways that directly or indirectly result in the generation of or metabolism of inositol phosphate.

As used herein, the terms “modulate” or “modulates” in reference to a receptor, enzyme or signaling pathway include any measurable alteration to the quality and/or quantity and/or intensity of signal generated, including, but not limited to, any measurable alteration to receptor or enzymatic activity. Modulation may occur via direct interaction of a compound with a receptor, enzyme, or other protein in the signaling pathway. Modulation can occur as the result of compounds interacting with any part of any protein, lipid, or carbohydrate moiety relevant to the signaling pathway. Modulation of receptor activity includes activation, inhibition and potentiation of the activation by an agonist (natural or otherwise) of the receptor. Modulation of the activity of an enzyme includes, but is not limited to, activation, enhancement, and inhibition of enzymatic activity. Modulation can also occur by compound interference with protein-protein interactions relevant to the signaling pathway.

As used herein, the terms “contact” or “contacting” refers to any method of combining and bringing-into contact various-components such as test compounds, cells, inositol, inositol phosphate, enzymes, or receptor agonists. For example, components can be brought into contact with cells by adding the components to the culture medium in a wide variety of culture vessels, tubes, plates, etc. Components can also be brought into contact in cell-free reaction solutions in a wide variety of reaction vessels, tubes, plates, etc.

As used herein, the term “increase” in reference to the amount of metal ion-bound inositol phosphate refers to any measurable augmentation of the amount of bound inositol phosphate.

As used herein, the term “decrease” in reference to amount of metal ion-bound inositol phosphate refers to any measurable diminution. of the amount of bound inositol phosphate.

The methods of the present invention can be used to monitor the activation of a receptor and/or its cognate pathway by a receptor agonist. The methods of the present invention can be used to test compounds for agonist activity at a receptor, i.e., to screen for compounds that function as agonists and activate a receptor and/or its cognate pathway. The methods of the present invention can be used to test compounds for their ability to act as antagonists of receptors and/or their cognate pathways.

In some embodiments, the methods comprise contacting the cells with labeled inositol, contacting the cells with a receptor agonist, whereby labeled inositol phosphate is generated, releasing the labeled inositol phosphate from the cells, contacting the labeled inositol phosphate with an immobilized metal ion under conditions permitting inositol phosphate to bind to the metal ion; and detecting labeled inositol phosphate bound to the immobilized metal ion, wherein bound labeled inositol phosphate is indicative of activation of the receptor and/or its pathway.

One aspect of the present invention is directed to methods of detecting the activation of phospholipase C-linked receptors and/or their pathways in cells expressing a receptor that utilizes a phospholipase C signaling pathway. These methods rely on the detection of inositol phosphate with immobilized metal ions to monitor the activity of a receptor and/or its cognate pathway.

Any cells in which a phospholipase C-linked receptor is expressed or can be engineered to be expressed can be used. Such cells include, but are not limited to, mammalian cells including, but not limited to, human, hamster, mouse, rat, or monkey, and non-mammalian cells such as amphibian (e.g., frog), fish cells, insect cells, and yeast cells.

Any receptor and/or pathway that causes or leads to the formation of inositol phosphate as a result of its activation can be assayed in the methods of the present invention. By way of non-limiting example, the methods of the present invention can be used to assay membrane-linked receptors and their cognate pathways that are linked to phospholipase C activation, including, but not limited to, members of the seven transmembrane domain G protein-coupled receptor superfamily, e.g., neurokinin NK1 receptor and muscarinic ml acetylcholine receptor, and members of the single transmembrane domain tyrosine kinase-linked receptor superfamily, e.g., PDGF receptor and NGF receptor.

In some embodiments of the present invention the receptor is a seven transmembrane domain G protein-coupled receptor or a single transmembrane domain tyrosine kinase-linked receptor. In some embodiments of the present invention, the receptor is selected from neurokinin NK1 receptor, muscarinic ml acetylcholine receptor, PDGF receptor, and NGF receptor.

A receptor agonist is any ligand that activates the receptor of interest. There are many known ligand-agonist/receptor pairs. By way of non-limiting example, Substance P is an agonist of the neurokinin NK1 receptor, and platelet-derived growth factor (PDGF) is an agonist of the PDGF receptor.

Any traceable, detectable, or measurable label may be used for labeling the inositol or inositol phosphate used in the methods of the present invention. The labels that can be used to label the inositol or inositol phosphate include, but are not limited to, radiolabels, fluorescent labels, chemiluminescent labels, enzymatic labels, immunogenic labels, and hapten labels (e.g., biotin, digioxin).

In some embodiments of the present invention, the label is selected from a radiolabel, a fluorescent label, a chemiluminescent label, an enzymatic label, an immunogenic label or a hapten label.

In some embodiments of the present invention, the label is a radiolabel.

Labels can be attached to inositol or inositol phosphate by any suitable methods known in the art. For example, the labels can be attached to the inositol or inositol phosphate covalently or non-covalently. The labels can also be attached to the inositol or inositol phosphate directly or indirectly via a linker. The labels can also be attached to the inositol or inositol phosphate via a cleavable linkage or linker, e.g., the linkage or linker that is cleavable via a physical, a chemical or an enzymatic treatment.

Releasing the inositol phosphate (including labeled inositol phosphate) from cells can be achieved by many methods known to those of skill in the-art, including, but not limited to, mixing or treating the cells with a hypotonic solution or a detergent, or sonication.

Any metal ion that will bind inositol phosphate can be used in the methods of the present invention. By way of non-limiting example, metal ions that can be used with the present invention include Zr⁴⁺, Ga³⁺, Al³⁺, Fe³⁺, Sc³⁺, and Lu³⁺, and mixtures thereof. Conditions permitting inositol phosphate to bind the aforementioned metal ions include a pH in the range of from about 2.0 to about 6.0.

In some embodiments of the present invention, the metal ion is selected from Zr⁴⁺, Ga³⁺, Al³⁺, Fe³⁺, Sc³⁺, and Lu³⁺, and mixtures thereof. In some embodiments of the present invention the metal ion is Zr⁴⁺.

Metal ions can be immobilized by affixing or otherwise attaching them to a solid support. For example, metal ions can be immobilized to an affinity matrix, which is a solid support having metal ion-chelating compounds covalently attached to it. Metal ion-chelating compounds include, but are not limited to, iminodiacetic acid (IDA), nitrilotriacetic acid (NTA), carboxymethylated aspartic acid (CM-Asp) and triscarboxymethyl ethylene diamine (TED). By way of non-limiting example the solid support can be agarose beads, sepharose beads, acrylic beads, plastic microtiter plates, polyvinyltoluene (pvt) plastic (such as scintillation proximity assay (SPA) beads (Amersham (Piscataway, N.J.) (Bosworth et al., 1989, Nature, 341:167-168; Alouani, 2000, Methods Mol. Biol., 138:135-41; Cook, 1996, Drug Discov. Today, 1:287-294), magnetic beads, fluorescent beads, or polystyrene (such as FlashPlate®). SPA beads and FlashPlate® have solid scintillant embedded in the plastic, which permits the measurement of a bound radioactive label without rinsing or removal of any unbound labeled material. SPA beads are highly sensitive and easy to use in 96-well or higher density format high throughput screening processed. FlashPlate® is a white polystyrene microplate designed for high-volume, in-plate radiobinding assays. The interior of each well is permanently coated with a thin layer of polystyrene-based scintillant that provides a platform for nonseparation assays using a variety of isotopes without the addition of liquid scintillation cocktail.

In some embodiments of the present invention, the metal ion is immobilized to SPA beads.

Labeled inositol phosphate can be detected by many methods known to those of skill in the art. Methods of detecting bound labeled inositol phosphate include, but are not limited to, radioactivity counting, light absorption, fluorescence or chemiluminescence measurement, and colorimetric measurement. The method of detection will depend on the type of label used and the type of solid support material. For example, if SPA beads or FlashPlate® are used as the solid support, radiolabeled inositol phosphate can be measured directly by using a scintillation counter (such as Topcount® NXT® or MicroBeta® Counter, Perkin-Elmer Life Sciences (Boston, Mass.). In other detection methods, unbound material may need to be removed prior to measurement of the bound labeled inositol phosphate. Methods of removal of unbound labeled material and retention of bound labeled material will be readily apparent to those of skill in the art, and include, but are not limited to, filtration or centrifugation.

In another aspect, the present invention is directed to methods for identifying compounds that modulate a signaling pathway. These methods are based upon the detection or measurement of inositol phosphate, using immobilized metal ion, in samples comprising a signaling pathway. These methods are carried out using signaling pathway samples generated in the presence and in the absence of a test compound. An alteration in the amount of bound inositol phosphate detected when the assay is carried out in the presence of a test compound identifies the test compound as a compound that modulates the signaling pathway.

In another aspect, the present invention is directed to methods for identifying compounds that modulate phospholipase C-linked receptor and/or phospholipase C-linked receptor pathway activation. These methods are carried out in the presence and in the absence of a test compound, and cells expressing a receptor that utilizes a phospholipase C signaling pathway are contacted with labeled inositol and with a receptor agonist, whereby labeled inositol phosphate is generated. The labeled inositol phosphate is released from the cells and contacted with an immobilized metal ion under conditions to allow inositol phosphate to bind to the metal ion. The bound labeled inositol phosphate is detected. An alteration in the amount of bound labeled inositol phosphate detected when the assay is carried out in the presence of a test compound identifies the test compound as a compound that modulates phospholipase C-linked receptor activation.

Another aspect of the present invention relates to methods for detecting inositol monophosphatase activity in a sample comprising contacting the sample with labeled inositol phosphate under conditions permitting inositol monophosphatase to hydrolyze phosphate from inositol phosphate, contacting the sample with an immobilized metal ion under conditions permitting inositol phosphate to bind to the metal ion, and detecting labeled inositol phosphate bound to the immobilized metal ion. A decrease in the amount of bound labeled inositol phosphate, as compared with a control, is indicative of inositol monophosphatase activity in the sample.

Detection of the presence of functionally active inositol monophosphatase in a sample is based upon the measurement of inositol monophosphatase activity in the sample using metal ions to bind labeled inositol phosphate for quantification of inositol monophosphatase-catalyzed hydrolysis of input labeled inositol phosphate. A decrease in the amount of labeled inositol phosphate detected in the output, or as compared to a negative control sample (without inositol monophosphatase activity), is indicative of the presence of inositol monophosphate activity in the sample.

Any sample may be tested for the presence of inositol monophosphatase enzyme and its activity. Samples may come from any source including, but not limited to, biological sources. Examples of biological samples that can be assayed with the methods of the present invention include, but are not limited to, cerebrospinal fluid, serum or tissue extracts.

Labels for the inositol phosphate include, but are not limited to, radiolabel, fluorescent label, chemiluminescent label, enzymatic label, immunogenic label, and hapten label.

Conditions that that allow inositol monophosphatase to catalyze the hydrolytic reaction to remove phosphate from the inositol phosphate are known to those of skill in the art and include, but are not limited to, a pH ranging from about 6.0 to about 8.0, and temperatures ranging from about 10° C. to about 40° C.

In some embodiments of the present invention, the hydrolysis reaction is terminated prior to contacting the sample with the immobilized metal ion.

Methods of terminating the reaction are known to those of skill in the art and include, but are not limited to, adding an acidic solution to render the pH of the reaction mixture in a range of about 2.0 to about 4.0.

Another aspect of the present invention relates to methods for identifying compounds that modulate inositol monophosphatase activity comprising, in the presence and in the absence of a compound, contacting inositol monophosphatase with labeled inositol phosphate under conditions permitting inositol monophosphatase to hydrolyze phosphate from inositol phosphate, contacting the reaction mixture with an immobilized metal ion under conditions permitting inositol phosphate to bind to the metal ion, and detecting labeled inositol phosphate bound to the immobilized metal ion, wherein an alteration in the amount of bound labeled inositol phosphate in the presence of a compound identifies said compound as a compound that modulates inositol monophosphatase activity.

Any form of functional inositol monophosphatase can be used in the methods of the present invention,-including, but not limited to, purified native enzyme, recombinantly expressed enzyme, and naturally occurring or genetically-manipulated mutant or variant forms of the enzyme, in any state of purity.

In some embodiments of the present invention, purified inositol monophosphatase is used.

In some embodiments of the present invention the hydrolysis reaction is terminated prior to contacting the sample with the immobilized metal ion.

In another aspect of the present invention, methods are provided for detecting inositol-1-phosphate synthase activity in a sample comprising contacting the sample with labeled inositol under conditions permitting inositol-1-phosphate synthase to catalyse addition of phosphate to inositol, contacting the sample with an immobilized metal ion under conditions permitting inositol phosphate to bind to the metal ion, and detecting labeled inositol phosphate bound to the immobilized metal ion; wherein an increase in the amount of bound labeled inositol phosphate as compared with a control is indicative of inositol-1-phosphate synthase in the sample.

Detection of the presence of inositol-1-phosphate synthase in a sample is based upon the measurement of inositol-1-phosphate synthase activity in the sample using metal ions to bind labeled inositol phosphate for quantification of inositol-1-phosphate synthase-catalyzed phosphorylation of input labeled inositol. An increase in the amount of labeled inositol phosphate detected in the output, or as compared to a negative control sample (without inositol-1-phosphate synthase activity), is indicative of the presence of inositol-1-phosphate synthase in the sample.

Any sample may be tested for the presence of inositol-1-phosphate synthase enzymatic activity. Samples may come from any source including, but not limited to, biological sources. Examples of biological samples that can be assayed with the methods of the present invention include, but are not limited to, cerebrospinal fluid, serum, and tissue extracts.

Labels for the inositol include, but are not limited to, radiolabel, fluorescent label, chemiluminescent label, enzymatic label, immunogenic label, and hapten label

Conditions that allow inositol-1-phosphate synthase to catalyze the reaction that adds a phosphate group to inositol to yield inositol phosphate are known to those of skill in the art and include, but are not limited to, a pH ranging from about 6.0 to about 8.0, and temperatures ranging from about 10° C. to about 40° C.

In some embodiments of the present invention, the kinase reaction is terminated prior to contacting the sample with the immobilized metal ion.

Methods of terminating the kinase reaction are known to those of skill in the art and include, but are not limited to, adding an acidic solution to render the pH of the reaction mixture in a range of about 2.0 to about 4.0.

Another aspect of the present invention relates to methods for identifying compounds that modulate inositol-1-phosphate synthase activity comprising, in the presence and in the absence of a compound, contacting inositol-1-phosphate synthase with labeled inositol under conditions permitting inositol-1-phosphate synthase to catalyse addition of phosphate to inositol, contacting the reaction mixture of with an immobilized metal ion under conditions permitting inositol phosphate to bind to the metal ion, and detecting labeled inositol phosphate bound to the immobilized metal ion, wherein an alteration in the amount of bound labeled inositol phosphate in the presence of a compound identifies said compound as a compound that modulates inositol-1-phosphate synthase activity.

Any form of functional inositol-1-phosphate synthase can be used in the methods of the present invention, including, but not limited to, purified native enzyme, recombinantly expressed enzyme, and naturally occurring or genetically-manipulated mutant or variant forms of the enzyme, in any state of purity.

In some embodiments of the present invention, purified inositol-1-phosphate synthase is used.

In some embodiments of the present invention, the kinase reaction is terminated prior to contacting the sample with the immobilized metal ion.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Materials and Methods

Materials

Myo-[³H]inositol (specific activity=110 Ci/mmol) and D-myo[³H]inositol-1-phosphate (specific activity=20 Ci/mmol) were purchased from Amersham Biosciences Corp. (Piscataway, N.J.) and American Radiolabeled Chemicals, Inc. (St. Louis, Mo.), respectively. ATP, formic acid, acetic acid, MES, MOPS, HEPES, LiCl, FeCl₃, CaCl₂, MgCl₂, NiCl₂, CuCl₂, substance P and L-733060 were from Sigma-Aldrich (St. Louis, Mo.). AlCl₃ was from Alfa Aesar (Ward Hill, Mass.). GaCl₃, ScCl₃, LuCl₃ and ZrOCl₂ were from Acros Organics (Morris Plains, N.J.). Human recombinant platelet-derived growth factor (PDGF-BB) was from Calbiochem (San Diego, Calif.). Swiss 3 T 3 cell line was obtained from ATCC (Manassas, Va.). The TopCount® NXT® Microplate Scintillation and Luminescence Counterinstrument was purchased from PerkinElmer Life Sciences, Inc. (Boston, Mass.).

Preparation of IMAC-SPA Beads

PVT SPA beads were custom coated with metal chelating compound, iminodiacetic acid (IDA), by Amersham Biosciences Corp. (Piscataway, N.J.). Metal ions were loaded onto the beads by re-suspending 1 gram of beads in 40 ml solution of 100 mM AlCl₃, FeCl₃, GaCl₃, ScCl₃, LuCl₃, or ZrOCl₂. After 15 min of gentle rocling at room temperature, the free metal ions were removed by spinning down the beads and washing the beads 4 times with de-ionized water. The loaded SPA beads were re-suspended at 20 mg/ml in water or 20 mM formic acid.

Characterization of Inositol Phosphate Binding to IMAC-SPA Beads

To test whether IMAC-SPA beads could entrap inositol phosphate, but not inositol, 1 mg (100 μl) IMAC-SPA beads loaded with different metal ions were mixed with 10 nCi of [³H]inositol (0.1 pmol) or [³H]inositol-1-phosphate (0.5 pmol) in each well of a 96-well, 350 ul Unifilter plate (Whatman). After 30 min of vigorous shaking, the binding mixtures were filtered using a Multiscreen vacuum manifold. The flow-through samples were collected in a 96-well white opaque plate and their radioactivity determined by liquid scintillation counting on TopCount® NXT® after adding 200 μl of Microscint. The radioactivity trapped on the beads was measured directly by SPA on TopCount® NXT®.

To test the pH effect on the binding of inositol phosphate to IMAC-SPA beads, the beads were re-suspended in 20 mM different buffers, including formate (pH 3.0), acetate (pH 4.0 and pH 5.0), MES (pH 6.0), MOPS (pH 7.0) and HEPES (pH 8.0), mixed with 10 nCi of [₃ H]inositol-1-phosphate for 5 min and the bound radioactivity quantified using TopCount® NXT®.

The binding capacity of Zr⁴⁺-SPA beads was determined by testing the ability of increasing concentrations of unlabeled myo-inositol-1-phosphate or ATP to block the binding of [³H]inositol-1-phosphate to the beads.

Cell Culture and Receptor Stimulation

CHO cells stably expressing human neurokiuin NK1 receptors were maintained in 5% CO₂ and at 37° C. in Ham's F12 medium supplemented with 10% fetal bovine serum (FBS) and 0.5 mg/ml G418. For experiments, the cells were plated onto 96-well plates in inositol-free DMEM medium containing 10% FBS and 5 μCi/ml [₃ H]inositol (0.5 μCi/well). After 16-48 hr incubation, the medium was removed and the cells were incubated for 15 min with or without NK1 antagonist in phosphate-free lithium-containing Hanks solution (PFLH) (composition: 20 mM HEPES, pH 7.4, 1.25 mM MgCl₂, 1.25 mM CaCl₂, 5 mM KCl, 125 mM NaCl, 10 mM LiCl and 10 mM glucose). The cells were then exposed to various concentrations of NK1 agonist, Substance P, for 45-60 min in PFLH solution at 37° C. At the end of the incubation, the agonist solution was removed and 100 μl ice-cold 20 mM formic acid solution (pH3.0) containing 2 mM myo-inositol was added in each well to release inositol phosphates from the cells. After incubation at 4° C. for over 10 min, the samples were transferred to a white opaque plate, and 100 μl of metal ion loaded IMAC-SPA beads added to each well. Alternatively, IMAC-SPA beads could be added directly to the cell plate to eliminate the sample transfer step. The amount of [³H] inositol phosphates generated in the cells was then determined by measurement of the radioactivity on the SPA beads on TopCount® NXT®.

Swiss 3 T 3 cells were maintained in DMEM medium supplemented with 10% heat-inactivated calf serum. For PI hydrolysis assay, the cells were grown in 96-well plates in [³H]inositol-containing medium for 16-48 hr, then deprived of serum for 24 hr. The cells were stimulated with PDGF-BB for 45-60 min and the inositol phosphate accumulated in the cells was measured by IMAC-SPA in the same way as described for NK1 receptors.

### Example 2

### Measurement of [³H]Inositol Phosphate Generated From PI Hydrolysis

Metal ions, immobilized on a solid support as an affinity matrix, were used to bind and isolate radiolabeled inositol phosphate, which was subsequently quantified by SPA technology. SPA beads are microspheres 5 microns in diameter consisting of a solid scintillant-containing polyvinyltoluene core coated with a polyhydroxy film (Cook, supra). A metal chelating compound, iminodiacetic acid, was covalently attached to the coating, allowing metal ions to be immobilized on the SPA beads. A phosphate moeity interacts with an immobilized metal ion through two coordination bonds and forms a strong four-member ring complex (Andersson et al., supra; Chaga et al., supra; Muszynska et al., supra; Hohnes et al., supra). The binding of [³H]inositol phosphates to the SPA beads via the interaction of their phosphate moeities with the immobilized metal ions brought the radioisotope in close proximity to the scintillant embedded in the beads and caused energy transfer and photon emission which were readily detected by TopCount® NXT® or MicroBeta® Reader (PerkinElmer Life Science).

### Example 3

### Interactions of Inositol Phosphate with IMAC-SPA Beads

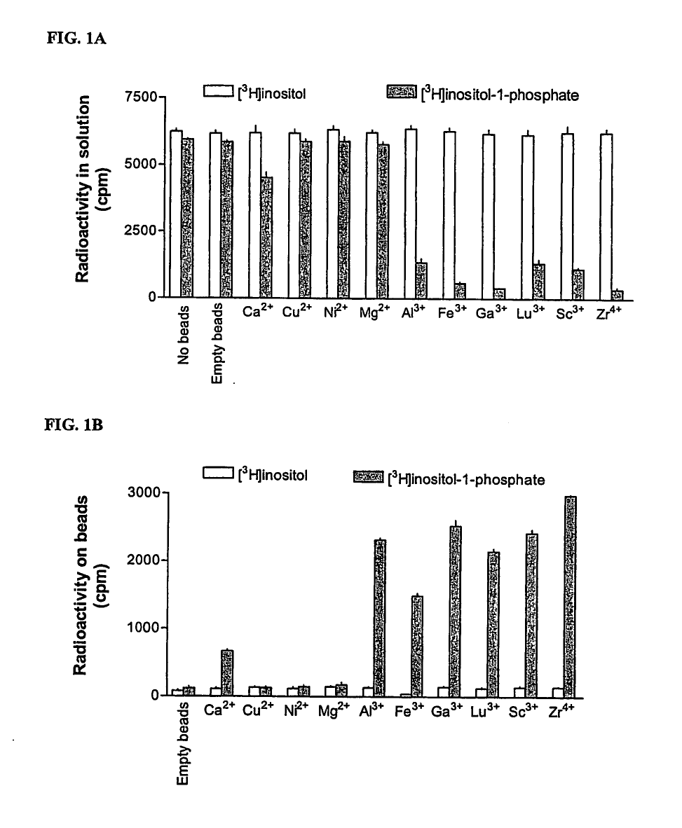

A number of metal ions can be immobilized on solid support via IDA groups (Sulkowski, supra; Yip et al., supra; Chaga, 2001, J. Biochem. Biophys. Methods, 49:313-334). Metal ions can be divided into three categories (hard, intermediate and soft) based on their preferential reactivity towards nucleophiles (Chaga, 2001, supra; Pearson, R., 1973, Hard and Soft Acids and Bases, pp 53-85, Hutchington &Ross, Stroudsburg, Pa.). The hard Lewis metal ions (Al³⁺, Ca²⁺, Fe³⁺, Lu³⁺, Sc³⁺, Zr⁴⁺) show preference for oxygen, while the soft metal ions (Cu+, Hg²⁺, Ag⁺) prefer sulfur. The intermediate (or transition) metal ions (Ni²⁺, Zn²⁺, Co²⁺) coordinate nitrogen, oxygen and sulfur. To identify the metal ions which could be used as an affinity ligand to selectively entrap inositol phosphate onto the SPA beads, we immobilized 10 different metal ions onto SPA beads, incubated the beads with [³H]inositol-1-phosphate or [³H]inositol and then used vacuum filtration to separate the bound from free inositides. As shown in FIG. 1, there was no significant binding of [³H]inositol to SPA beads loaded with any of the metal ions. The amount of inositol radioactivity retained on the beads was at background level and was independent of the metal ions used. In contrast to inositol, the amount of free ([³ H]inositol-1-phosphate in the flow-through samples was reduced by various degrees depending on the metal ions that were immobilized on the beads. The loss of radioactivity in the flow-through corresponds to the retention on the SPA beads. Immobilized hard metal ions Al³⁺, Fe³⁺, Ga³⁺, Lu³⁺, Sc³⁺ and Zr⁴⁺ adsorbed 80-90% and Ca²⁺ adsorbed 20% of [³H+) inositol-1-phosphate, while -the transition metal ions Cu²⁺ and Ni²⁺ did not adsorb a significant amount. The radioactivity of [³H]inositol-1-phosphate trapped on the beads was measured by SPA technology which had a ˜50% lower efficiency than liquid scintillation counting, therefore the total cpm in the SPA samples underestimated the amount of radioactivity that was adsorbed. Among the metal ions used, only Ni²⁺, Cu²⁺ and Fe³⁺ have colors. Due to the color quenching effect, the radioactivity on the Fe³⁺-loaded SPA beads was much lower than that on the Zr⁴⁺-loaded beads, although the amount of [³H]inositol-1-phosphate retained on the beads were very similar. These data indicate that several hard metal ions, including Zr⁴⁺, Al³⁺, Fe³⁺, Ga³⁺, Lu³⁺ and Sc³⁺, can be immobilized on the SPA beads and utilized as affinity ligands to entrap and quantify [³H]inositol phosphates in the solution.

### Example 4

### Effect of pH on Inositol Phosphate Binding to IMAC-SPA Beads

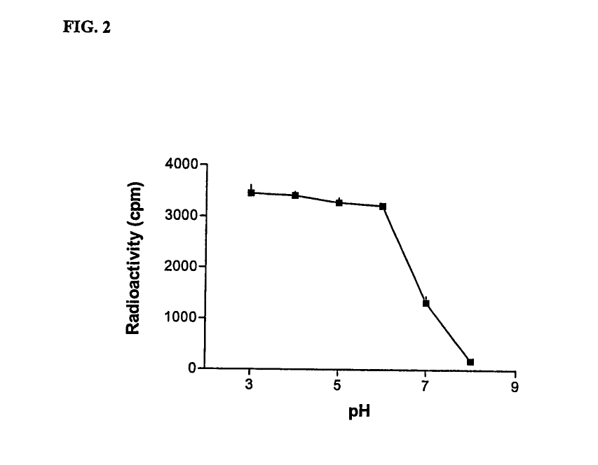

To evaluate the effect of pH on the assay, the binding of [³H]inositol-1-phosphate to Zr⁴⁺-SPA beads was carried out in solutions containing 20 mM buffer at different pH ranging from 3.0 to 8.0. As shown in FIG. 2, at pH 6.0 or below, the binding was optimal. At pH 7.0, the binding was significantly reduced, and at pH 8.0, the binding was almost completely abolished. Since the binding of metal ions to IDA and the SPA counting efficiency are not affected by neutral pH (Cook, 1996, supra; Chaga, 2001, supra; Porath 1992, Protein Expr. Purif., 3:263-281), it is likely that the protonation status of the phosphate group, which has a pKa at ˜7.2, affects the binding of [³H]inositol-1-phosphate to the immobilized metal ions. 20 mM formic acid (pH3.0) effectively released inositol phosphates from the cells after stimulation for measurement of PI hydrolysis by IMAC-SPA.

### Example 5

### Assessment of Binding Capacity of Zr⁴⁺-SPA Beads

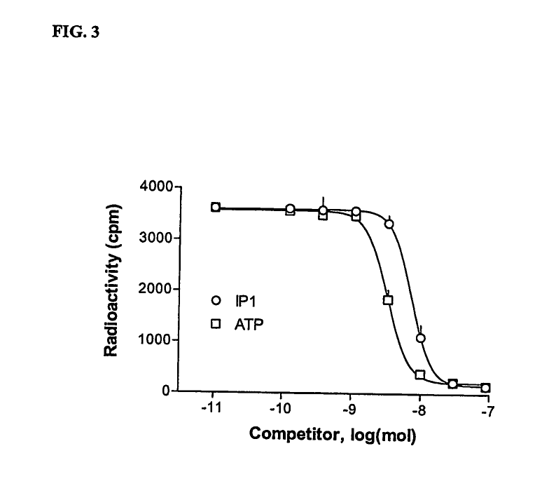

To determine the binding capacity of Zr⁴⁺-SPA beads, the binding of 10 nCi [³H]) inositol-1-phosphate to 2 mg of the beads, pre-incubated with increasing concentrations of unlabeled inositol phosphate or ATP, was measured. As shown in FIG. 3, pre-treatment of 2 mg beads with up to 1 nmol unlabeled inositol phosphate or ATP did not block the binding of [³H]inositol-1-phosphate to Zr⁴⁺-SPA beads, indicating the binding capacity is at ˜0.5 nmol/mg beads.

### Example 6

### Measurement of NK1-Mediated Response

The neurokinin NK1 receptor is a member of the seven-transmembrane-domain G protein coupled receptor superfamily. Through the coupling of Gq/11 class of heterotrimeric G protein, stimulation of NK1 receptor by agonists, such as Substance P, triggers the activation of PLC-β and results in an increase in PI hydrolysis (Severini et al., 2002, Pharmacol. Rev., 54:285-322; Seabrooka et al., 1996, Eur. J. Pharmacol., 317:129-135).

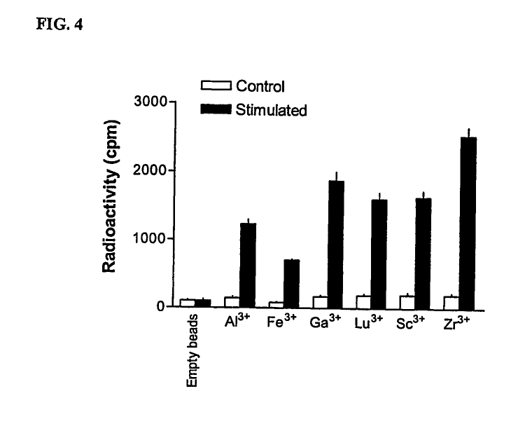

To test whether metal ion-loaded SPA beads can be used to measure NK1-mediated PI hydrolysis, CHO cells, stably expressing NK1 receptor, were incubated with [³H]inositol and stimulated with Substance P in the presence of 10 mM LiCl. The acid-soluble components of the cells were then released into 20 mM formic acid solution and mixed with SPA beads, loaded with Hard Lewis metal ions, to measure the [³H]inositol phosphates generated from PI hydrolysis. FIG. 4 depicts the radioactivity released from cells treated with saline (control) or 0.1 μM Substance P as detected by the SPA beads with immobilized Al³⁺, Fe³⁺, Ga³⁺, Lu³⁺, Sc³⁺ and Zr⁴⁺. Significant increases in [³H]inositol phosphate production in the cells stimulated with Substance P were detected by all six immobilized hard Lewis metal ions. The Zr⁴+-loaded SPA beads gave the best performance, yielding the highest cpm and a 12-fold increase in signal over the control.

### Example 7

### Optimization of Amount of SPA Beads

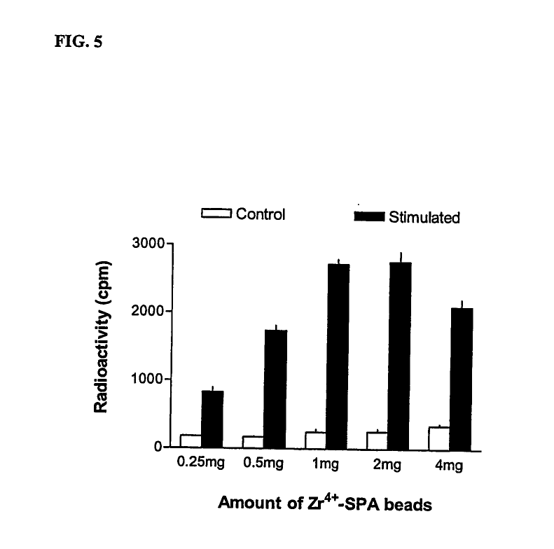

To optimize the assay conditions, different amounts of SPA beads were added to the wells of a 96-well plate to measure Substance P-stimulated PI hydrolysis. As shown in FIG. 5, the best results can be achieved by using 1 mg/well or 2 mg/well Zr⁴⁺-loaded SPA beads. The lower cpm with 4 mg/well bead was probably due to the stacking effect of the beads that blocked the light path. To further simplify the assay procedures, the cells were plated on white opaque 96-well plates, and Zr⁴⁺-SPA beads in 20 mM formic acid (pH3.0) were added directly to the cell plate after receptor stimulation. However, elimination of the transfer step resulted in only a 2-fold increase in background and 2-fold reduction of the assay window.

### Example 8

### Evaluation of Functional Properties of NK1 Receptor Agonist and Antagonist Using Zr⁴⁺-SPA Beads

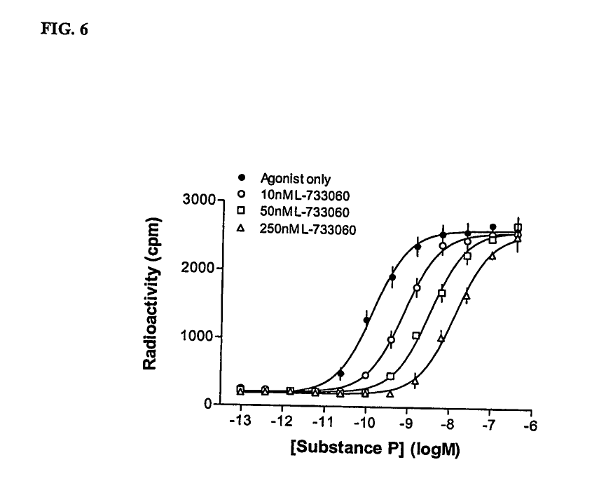

To ensure that the measurement of PI hydrolysis by this new approach does not change the receptor pharmacology, we evaluated the functional properties of a NK1 receptor agonist and antagonist using Zr⁴⁺-SPA beads. FIG. 6 depicts the concentration-response curves of Substance P-induced PI hydrolysis with or without the pre-treatment of the cells with NK1 antagonist L-733060. Substance P stimulated PI hydrolysis in NK1 CHO cells in a concentration-dependent manner with an EC₅₀ value of 0.15 nM. L-733060 inhibited competitively the PI hydrolysis induced by Substance P and shifted the concentration-response curve to the right. From Schild regression analysis, the functional potency (pKa) of the antagonist was determined to be close to 9.0, which is consistent to the values reported in the literature (Seabrooka et al., supra; Noailles et al., 2002, Ann. NY Acad. Sci., 965:267-73; Rupniak et al., 2000, Neuropharmacology, 39:1413-1421).

### Example 9

### Measurement of PDGF Receptor-Mediated Response

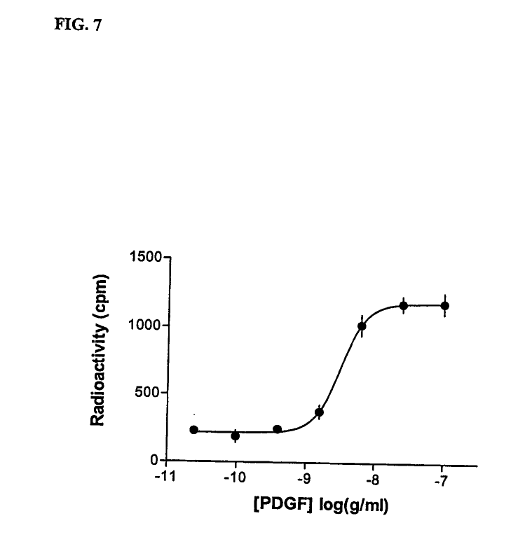

The PDGF receptor belongs to the superfamily of tyrosine kinase-linked growth factor receptors. Stimulation of the PDGF receptor leads to activation of PLC-y through Ras-related GTPase (Ji et al., 1999, Mol. Cell. Biol., 1999. 19:49614970; Wang et al., 1998, Mol. Cell. Biol., 18:590-597; Heldin et al., 1998, Biochim. Biophys. Acta, 1378:F79-113; Stice et al., 1999, Front. Biosci., 4:D72-86). To demonstrate the use of immobilized metal ions on SPA beads for the measurement of PI hydrolysis mediated by growth factor receptors, quiescent Swiss 3T 3 cells were stimulated with PDGF-BB, and the amount of [³H]inositol phosphates generated in the cells was determined using Zr⁴⁺-SPA beads. As shown in FIG. 7, PDGF stimulated PI hydrolysis in a concentration-dependent manner with a maximal 5-fold increase in inositol phosphate production and an EC₅₀ value of 3.0 ng/ml. The EC₅₀ value determined by this new approach was not significantly different from the values reported in the literature (Berridge et al., 1984, supra; Blakeley et al., 1989, Biochem. J., 258:177-85; Chu et al., 1985, J. Cell. Physiol., 124:391-396).

The foregoing examples are meant to illustrate the invention and are not to be construed to limit the invention in any way. Those skilled in the art will recognize modificationis that are within the spirit and scope of the invention.

We claim:

1. A method for detecting or measuring inositol phosphate in a sample comprising
a) contacting the sample with an immobilized metal ion; and
b) detecting inositol phosphate as bound to the immobilized metal ion.

2. The method of claim 1, wherein the metal ion is selected from Zr⁴⁺, Ga³⁺, Al³⁺, Fe³⁺, Sc³⁺, and Lu³⁺, and mixtures thereof

3. The method of claim 2, wherein the metal ion is Zr⁴⁺.

4. The method of claim 1, wherein the metal ion is immobilized to scintillation proximity assay (SPA) beads.

5. The method of claim 1, wherein the inositol phosphate is attached to a label.

6. The method of claim 5, wherein the label is selected from radiolabel, fluorescent label, chemiluminescent label, enzymatic label, immunogenic label, and hapten label.

7. The method of claim 6, wherein the label is a radiolabel.

8. A method for detecting or measuring inositol phosphate in a sample comprising
a) contacting the sample with an immobilized metal ion bound to inositol phosphate, said inositol phosphate attached to a label; and
b) detecting displacement of the inositol phosphate from the metal ion.

9. The method of claim 8, wherein the metal ion is selected from Zr⁴⁺, Ga³⁺, Al³⁺, Fe³⁺, Sc³⁺, and Lu³⁺, and mixtures thereof.

10. The method of claim 9, wherein the metal ion is Zr⁴⁺.

11. The method of claim 8, wherein the metal ion is immobilized to SPA beads.

12. The method of claim 8, wherein the label is selected from radiolabel, fluorescent label, chemiluminescent label, enzymatic label, immunogenic label, and hapten label.

13. The method of claim 12, wherein the label is a radiolabel.

14. A method for detecting or measuring activation of a signaling pathway comprising detecting inositol phosphate in accordance with claim 1 or claim 8.

15. A method for detecting or measuring modulation of a signaling pathway comprising detecting inositol phosphate in accordance with claim 1 or claim 8.

16. A method for identifing compounds that modulate a signaling pathway comprising, in the presence and in the absence of a test compound, detecting inositol phosphate in accordance with claim 1 or claim 8.

17. A method for detecting or measuring activation of a signaling pathway comprising
a) contacting a sample with an immobilized metal ion; and
b) detecting inositol phosphate as bound to the immobilized metal ion.

18. A method for detecting or measuring modulation of a signaling pathway comprising
a) contacting a sample with an immobilized metal ion; and
b) detecting inositol phosphate as bound to the immobilized metal ion.

19. A method for identifying compounds that modulate a signaling pathway comprising, in the presence and in the absence of a test compound
a) contacting the sample with an immobilized metal ion; and
b) detecting inositol phosphate as bound to the immobilized metal ion.

20. A method for detecting activation of a phospholipase C-linked receptor and/or its pathway comprising:
a) providing cells expressing a receptor that utilizes a phospholipase C signaling pathway;
b) contacting the cells with labeled inositol;
c) contacting the cells with a receptor agonist, whereby labeled inositol phosphate is generated;
d) releasing the labeled inositol phosphate from the cells;
e) contacting the labeled inositol phosphate with an immobilized metal ion under conditions permitting inositol phosphate to bind to the metal ion; and
f) detecting labeled inositol phosphate as bound to the immobilized metal ion;
wherein bound labeled inositol phosphate is indicative of receptor and/or pathway activation.

21. The method of claim 20, wherein the receptor is a seven transmembrane domain G protein-coupled receptor or a single transmembrane domain tyrosine kinase-linked receptor.

22. The method of claim 21, wherein the receptor is selected from neurokinin NK1 receptor, muscarinic ml acetylcholine receptor, PDGF receptor, and NGF receptor.

23. The method of claim 20, wherein the metal ion is selected from Zr⁴⁺, Ga³⁺, Al³⁺, Fe³⁺, Sc³⁺, and Lu³⁺, and mixtures thereof

24. The method of claim 23, wherein the metal ion is Zr⁴⁺.

25. The method of claim 20, wherein the metal ion is immobilized to SPA beads.

26. The method of claim 20, wherein the inositol phosphate label is selected from radiolabel; fluorescent label, chemiluminescent label, enzymatic label, immunogenic label, and hapten label.

27. The method of claim 26, wherein the inositol phosphate label is a radiolabel.

28. A method for identifying compounds that modulate a phospholipase C-linked receptor and/or its pathway comprising, in the presence and in the absence of a compound,
a) providing cells expressing a receptor that utilizes a phospholipase C signaling pathway;
b) contacting the cells with labeled inositol;
c) contacting the cells with a receptor agonist, whereby labeled inositol phosphate is generated;
d) releasing labeled inositol phosphate from the cells;
e) contacting the labeled inositol phosphate with an immobilized metal ion under conditions to allow inositol phosphate to bind to the metal ion; and
f) detecting labeled inositol phosphate as bound to the immobilized metal ion;
wherein an alteration in the amount of bound labeled inositol phosphate in the presence of a compound identifies said compound as a compound that modulates the phospholipase C-linked receptor and/or its pathway.

29. A method for detecting inositol monophosphatase activity in a sample comprising
a) contacting the sample with labeled inositol phosphate under conditions permitting inositol monophosphatase to hydrolyze phosphate from inositol phosphate;
b) contacting the sample with an immobilized metal ion under conditions permitting inositol phosphate to bind to the metal ion; and
c) detecting labeled inositol phosphate as bound to the immobilized metal ion;
wherein a decrease in the amount of bound labeled inositol phosphate, as compared to a control, is indicative of inositol monophosphatase activity in the sample.

30. The method of claim 29, wherein the hydrolysis reaction is terminated prior to contacting the sample with the immobilized metal ion.

31. A method for identifying compounds that modulate inositol monophosphatase activity comprising, in the presence and in the absence of a compound,
a) contacting inositol monophosphatase with labeled inositol phosphate under conditions permitting inositol monophosphatase to hydrolyze phosphate from inositol phosphate;
b) contacting the reaction mixture of step a) with an immobilized metal ion under conditions permitting inositol phosphate to bind to the metal ion; and
c) detecting labeled inositol phosphate as bound to the immobilized metal ion;
wherein an alteration in-the amount of bound labeled inositol phosphate in the presence of a compound identifies said compound as a compound that modulates inositol monophosphatase activity.

32. The method of claim 31, wherein the hydrolysis reaction is terminated prior to contacting the sample with the immobilized metal ion.

33. A method for detecting inositol-1-phosphate synthase activity in a sample comprising
a) contacting the sample with labeled inositol under conditions permitting inositol-1-phosphate synthase to catalyse addition of phosphate to inositol;
b) contacting the sample with an immobilized metal ion under conditions permitting inositol phosphate to bind to the metal ion; and
c) detecting labeled inositol phosphate as bound to the immobilized metal ion;
wherein an increase in the amount of bound labeled inositol phosphate, as compared to a control, is indicative of inositol-1-phosphate synthase activity in the sample.

34. The method of claim 33, wherein the kinase reaction is terminated prior to contacting the sample with the immobilized metal ion.

35. A method for identifying compounds that modulate inositol-1-phosphate synthase activity comprising, in the presence and in the absence of a compound,
a) contacting inositol-1-phosphate synthase with labeled inositol under conditions permitting inositol-1-phosphate synthase to catalyse addition of phosphate to inositol;
b) contacting the reaction mixture of step a) with an immobilized metal ion under conditions permitting inositol phosphate to bind to the metal ion; and
c) detecting labeled inositol phosphate as bound to the immobilized metal ion;
wherein an alteration in the amount of bound labeled inositol phosphate in the presence of a compound identifies said compound as a compound that modulates inositol-1-phosphate synthase activity.

36. The method of claim 35, wherein the kinase reaction is terminated prior to contacting the sample with the immobilized metal ion.
